(19) United States
(12) Patent Application Publication
(10) Pub. No.: US 2012/0207825 A1
(43) Pub. Date: Aug. 16, 2012

(54) PHARMACEUTICAL COMPOSITIONS FOR REDUCING ALCOHOL-INDUCED DOSE DUMPING

(76) Inventors: Sunilendu Bhushan Roy, Gujarat (IN); Sushrut Krishnaji Kulkarni, Gujarat (IN); Maulik Kiritkumar Panchal, Gujarat (IN); Kartik Yogeshkumar Shah, Gujarat (IN)

(21) Appl. No.: 13/393,658

(22) PCT Filed: Sep. 9, 2010
(86) PCT No.: PCT/IN2010/000604

§ 371 Date: May 2, 2012

(30) Foreign Application Priority Data
Sep. 17, 2009 (IN) 2154/MUM/2009

(51) Int. Cl. A61K 9/00; A61K 38/43; A61K 31/381; A61K 31/137; A61K 31/485; A61K 9/20; A61K 9/48; A61K 9/14; A61K 9/50; A61P 29/00; A61P 25/20; A61P 31/00; A61P 3/10; A61P 9/12; A61P 19/10; A61P 7/02; A61P 31/12; A61P 31/10; A61P 25/22; B05D 1/36; A61K 31/56

(52) U.S. Cl. 424/452; 424/400; 514/169; 424/94.1; 514/438; 514/649; 514/282; 424/465; 424/489; 424/490; 427/2.14

(57) ABSTRACT

A pharmaceutical composition is disclosed. The composition comprises a core comprising an active substance or a salt thereof; a separating layer comprising at least one sugar; and a functional layer comprising at least one pharmaceutically acceptable polymer, wherein the composition is resistant to dose dumping in presence of alcohol.

### FIELD OF THE INVENTION

The invention relates to controlled release pharmaceutical compositions, which resist alcohol-induced dose dumping and related side effects in presence of alcohol. In Particular, the composition comprises a sugar in a separating layer between a drug core and a functional layer. The invention also relates to processes for the preparation of such compositions.

### BACKGROUND OF THE INVENTION

The value of controlled release pharmaceutical compositions for the administration of a drug is well known. In particular, they provide a better cover of the therapeutic need since the useful plasma drug concentration can be maintained longer than in the case of immediate release compositions. In addition, they make it possible to avoid or limit the magnitude and number of peaks of excessive plasma drug concentration, thereby reducing the toxicity of the drug and its side effects. Furthermore, by virtue of their increased duration of action, these systems make it possible to limit the number of daily dosage units, thus reducing constraint for the patient and improving compliance with the treatment.

Controlled release drug compositions often contain higher amounts of drugs than immediate release compositions. Functionality and safety of a controlled release composition are based on a known controlled rate of drug release from the composition over an extended period of time after administration, such as 8-24 hours.

The relatively high amount of drug that is present in a controlled release composition can, in some instances, harm a patient if the composition releases the drug at a rate that is faster than the intended controlled release rate. In most cases, failure of a controlled release composition results in a rapid release of the drug into the blood stream. This rapid release is generally faster than the intended controlled release of the drug from the composition, and is sometimes referred to as “dose dumping”. Dose dumping can create severe consequences for a patient, including permanent harm and even death.

Oral pharmaceutical dosage forms are often taken with a commonly available beverage, such as water, juice, a carbonated beverage or occasionally an ethanol-containing beverage. An ethanol-containing beverage is commonly referred to as an alcoholic beverage, liquor, or simply alcohol. As used herein, “alcohol” refers to ethanol, or an ethanol-containing (“alcoholic”) beverage such as beer, wine, and hard liquors such as vodka, rum, or whiskey. Dose dumping in the presence of ethanol creates a safety concern because of the likelihood that a patient will ingest the composition with an alcoholic beverage. This can be exacerbated where the drug may interact with the alcohol.

Recent studies have shown that the presence of alcohol can accelerate the release of a drug contained in a controlled release pharmaceutical dosage form. In a first analysis, this alcohol effect can be explained by a degradation of the controlled-release system or by a modification of the solubility of the drug in the presence of a significant amount of alcohol. This situation is all the more likely to be encountered—and the consequences are likely to be all the more serious—if a large amount of alcoholic drink is ingested, if the drink has a high alcoholic strength and if the subject has an empty stomach. In practice, therefore, the ingestion of alcohol concomitantly with the administration of a controlled release pharmaceutical dosage form can result in the accelerated and potentially dangerous release of the drug in the patient. Depending on the type of drug, this accelerated release of the drug at best renders the controlled release pharmaceutical dosage form totally ineffective, and at worst jeopardizes the patient's vital prognosis.

In 2005, several drugs were either withdrawn from the market or had their warning labels enhanced because of the effects of ethanol on the controlled release compositions of the drug. FDA has also indicated that for all future sustained release products, in vitro testing for alcohol-induced undermining of sustained release characteristics may be advisable as a routine characterization test. Furthermore, FDA's position is that for certain drugs (e.g., drugs with a narrow therapeutic index or having consequences of high Cₘₐₓ or low Cₘᵢₙ), alcohol sensitive sustained release compositions should not be approved. FDA prefers that compositions be made ethanol-resistant by design, rather than simply a confirmation that dose dumping does not occur through an in vivo study. (c.f. Summary of FDA's position on alcohol-induced dose dumping as presented at the Pharmaceutical Sciences Advisory Committee Meeting Oct. 26, 2005)

US Publication No. 2006/0193912 describes compositions with a mixture of gums and ionizable gel strength enhancing agent that are expected to exhibit reduced alcohol induced dose dumping.

US Publication No. 2007/0264346 provides an oral pharmaceutical form comprising micromultiparticles of the reservoir type for the modified release of at least one active principle, said form being resistant to immediate dumping of the dose of active principle in the presence of alcohol.

International (PCT) Publication No. 2007/053698 describes once-a-day controlled release compositions of opioid analgesics that exhibit improved properties with respect to co-administration with aqueous alcohol.

International (PCT) Publication No. 2008/086804 describes use of polyglycol, especially, polyethylene glycol for the preparation of a pharmaceutical composition, wherein the composition is without ethanol induced dose dumping. According to the application, the oral solid dosage forms are prepared by heating in order to melt or soften the polymer followed by solidification.

US Publication No. 2008/0085304 describes ethanol-resistant controlled release pharmaceutical compositions comprising a hydrophilic gum, a homopolysaccharide gum, and a pharmaceutical diluent.

US Publication No. 2009/0155357 discloses a modified release oral dosage form comprising alcohol insoluble coating, which is preferably water insoluble.

The problem of dose dumping in the presence of alcohol has not yet been solved satisfactorily. Accordingly, there exists a need in the art to provide an extended release oral dosage form which has reduced potential for alcohol induced dose dumping. The inventors have surprisingly found that by introducing sugar in a separating layer between a core and an extended release or a functional layer, there is a reduction of potential for alcohol induced dose dumping.

### SUMMARY OF THE INVENTION

In one general aspect there is provided a pharmaceutical composition comprising a core comprising an active substance or a salt thereof; a separating layer comprising at least one sugar; and a functional layer comprising at least one pharmaceutically acceptable polymer, wherein the composition is resistant to dose dumping in presence of alcohol.

In another general aspect there is provided a pharmaceutical composition comprising a core comprising an active substance or a salt thereof; a separating layer comprising at least one sugar; and a functional layer comprising at least one pharmaceutically acceptable polymer, wherein the composition is resistant to dose dumping in presence of alcohol and wherein less than 40% of the active ingredient is released from the composition after 60 minutes in the presence of 40% alcohol at pH 1.2.

Embodiments of the pharmaceutical composition may include one or more of the following features. For example, the separating layer may include a binder and an anti-tacking agent in addition to sugar.

The sugar, binder and an anti-tacking agent in the separating layer may be present in a ratio of from 80:10:10 to 20:70:10.

The pharmaceutical composition may include one or more pharmaceutically acceptable excipients selected from diluents, disintegrants, binders, lubricants, glidants, plasticizers, anti-tacking agents, opacifying agents, and the like.

In another general aspect there is provided a process for preparing a pharmaceutical composition, the process comprising:
(i) preparing a core comprising an active substance or a salt thereof;
(ii) applying a separating layer comprising at least one sugar on the core;
(iii) applying a functional layer comprising at least one pharmaceutically acceptable polymer on the coated core of step (ii); and
(iv) converting into a suitable finished dosage form.

Embodiments of the pharmaceutical composition may include one or more of the following features. For example, the pharmaceutical composition may include one or more pharmaceutically acceptable excipients selected from diluents, disintegrants, binders, lubricants, glidants, plasticizers, anti-tacking agents, opacifying agents, and the like.

The details of one or more embodiments of the invention are set forth in the description below. Other features, objects and advantages of the invention will be apparent from the description and claims.

### BRIEF DESCRIPTION OF THE DRAWINGS

FIG. 1: Graphical illustration of effect of sucrose containing barrier layer on drug release

FIG. 2: Graphical illustration of effect of ratio of Sucrose:HPMC:Talc in the barrier layer on drug release

FIG. 3: Graphical illustration of effect of sucrose and lactose in the barrier layer on drug release (for 75% weight gain)

FIG. 4: Graphical illustration of effect of sucrose and lactose in the barrier layer on drug release (for 200% weight gain)

### DETAILED DESCRIPTION OF THE INVENTION

The inventors of the present invention have surprisingly found that addition of at least one sugar in a pharmaceutical composition can reduce dose dumping of a drug from the composition in presence of alcohol. In particular, the invention provides extended release (modified release, delayed release, or a combination thereof) pharmaceutical compositions and method for preventing alcohol induced dose dumping in those compositions. The pharmaceutical compositions described herein are more resistant to dose dumping compared to conventional extended release formulations, which make them more rugged, safer and applicable to a wide variety of drugs. The inventors have surprisingly found that the percentage of active substance released in initial hours from the composition as per the invention is significantly reduced even in presence of alcohol.

The invention further provides methods for preparing pharmaceutical compositions which give alcohol resistance and methods for increasing drug safety and reducing the potential for drug abuse. The alcohol resistant drug compositions are safer and have less potential for abuse when compared to commercially available compositions because their extended release dissolution profile in an aqueous solution or in an alcohol containing solution is essentially the same. The alcohol resistant pharmaceutical compositions described herein do not dose dump in the presence of alcohol.

An active substance or a drug used in the invention is the drug, which is prone to dose dumping in presence of alcohol irrespective of the solubility of the drug. The drug may be selected from the therapeutic category of drugs like anti-inflammatory agents, sedatives, hypnotics, antibiotics, antidiabetics, antihypertensives, anti-osteoporosis agents, antithrombotic agents, antivirals, antifungals, anticholinergic agents, anxiolytic agents, adrenergics, antipsychotics, anti-parkinsonism agents, anti convulsants, antiepileptics, CNS stimulants, antianginal agents, antiarrhythmics, anti-hyperlipidemic drugs, diuretics, antiasthmatics, anticoagulants, antianemia agents, vitamins, hormones, antihistaminics, anticancer agents, antiallergics, antiarthritis agents, antialzheimers' agents, vasopressin antagonists, anticonvulsants, steroids, an esthetics, thrombolytics, antacids, proton pump inhibitors, protease inhibitors, platelet aggregation inhibitors, mucolytics, antimalarials, antiemetics, laxatives, expectorants, enzymes, contraceptives, bronchodilators, antitussives, antimigraine agents, anthelmintics, and anorexiants.

The drugs may be selected from venlafaxine, duloxetine, cyclobenzaprine, quetiapine, trospium, propranolol, morphine, oxycodone, oxymorphone, amlodipine, hydrocodone, diazepam, paracetamol (acetaminophen), aspirin, ciprofloxacin, dicyclomine, celecoxib, alendronate, diacerein, acyclovir, fluconazole, epinephrine, divalproex, methylphenidate, metoprolol, fenofibrate, hydrochlorothiazide, montelukast, heparin, warfarin, hemoglobin, iron, ascorbic acid, leutinizing hormone, bicalutamide, donepezil, tolvaptan, cortisones, lidocaine, calcium carbonate, saquinavir, bromhexine, promethazine, bisacodyl, pancreatin, ethinyl estradiol, salbutamol, diphenhydramine, sumatriptan, diclofenac, metronidazole, orlistat, ibuprofen, indomethacin, ketorolac, tramadolol, oxcarbazepine, pioglitazone, rosiglitazone, miglitol, vildagliptin, sitagliptin, repaglinide, voglibose, alprazolam, chlorpromazine, cimetidine, pseudoephedrine, naproxen, piroxicam, atenolol, benazepril, captopril, lisinopril, fosinopril, enalapril, furosemide, indapamide, atenolol, felodipine, verapamil, cartenolol, carvedilol, cerivastatin, diltiazem, fluvastatin, irbesartan, candesartan, methyldopa, reserpine, bupropion, fluoxetine, paroxetine, escitalopram, sertraline, amitryptiline, imipramine, fexofenadine, clopidogrel, entacapone, levodopa, carbidopa, levetiracetam, lisinopril, losartan, lovastatin, niacin, pravastatin, ramipril, simvastatin, atorvastatin, valsartan, telmisartan, sildenafil, tadalafil, vardenafil, esomeprazole, famotidine, omeprazole, pantoprazole, rabeprazole, ranitidine, simethicone, artesunate, amodiaquine, benazepril, misoprostol, metformin, glipizide, and their pharmaceutically acceptable salts.

The “core” may refer to anything which is present below the separating layer. For example, the core may comprise one or more of non-pareil seeds, pellets, beads, granules, mini-tablets or a micro-tablet. The non-pareil seeds may be of any pharmaceutically acceptable excipients such as starch, sugar, microcrystalline cellulose, vegetable gums, waxes, and the like. The non-pareil seed may include starch and sugar. The inert core may be prepared by techniques such as granulation or extrusion-spheronization. For example, the inert core may be prepared by mixing one or more pharmaceutically acceptable excipients, moistening the mixture with water or a solvent, granulating and subsequently drying to obtain granules (inert cores), which granules are coated with an active substance to obtain the core. The inert core may also be prepared by mixing one or more pharmaceutically acceptable excipients, wetting with water or organic solvent and mixing in a high shear granulator to form a homogeneous wet mass, extruding the wet mass to form extrudates which are subsequently spheronized to form spheres (inert cores), which spheres are coated with an active substance to obtain the core. The core may be present in an amount ranging from 10% to 90% by weight of the composition. The core may also be prepared with the techniques known to a person skilled in the art, such as direct compression, wet granulation, dry granulation, and the like. The active substance may be mixed and/or granulated with the excipients to prepare the core.

The “separating layer” or a “barrier layer” may be present between the core and the functional layer. The separating layer may prevent direct contact of the components of the core and the rate-controlling polymer in the functional layer. The separating layer may also act to modify the sustained release of the drug. The separating layer as described herein includes at least one sugar, saccharide or a sugar alcohol. It may also include a binder and an anti-tacking agent. The ratio of the sugar to binder to anti-tacking agent in the separating layer may be from 80:10:10 to 20:70:10. The words “separating layer” and “barrier layer” may be used interchangeably as per the invention. The separating layer may be present in the composition for about 30-200% of weight gain on the core depending on the need.

In general, the sugar, saccharide or sugar alcohol used is soluble in water, and is partially soluble or insoluble in alcohol used. Suitable sugar, saccharide or sugar alcohol may include one or more of sucrose, lactose, dextrin, dextrose, fructose, glucose, mannitol, sorbitol, trehalose, xylitol, isomalt, maltitol, inositol, lactitol or a combination thereof.

The “functional layer” may include the layer that changes or modifies the release of the drug from the dosage form. It may be selected from delayed release layer, sustained release layer or combinations thereof. The layer comprises one or more rate-controlling polymers and optionally one or more pharmaceutically acceptable excipients such as plasticizer, anti-tacking agent and opacifying agent.

The functional layer may include one or more rate-controlling polymers selected from hydrophilic polymers, hydrophobic polymers or combinations thereof. The rate-controlling polymer used in the sustained release layer may include one or more of cellulosic polymers such as ethylcellulose, hydroxypropyl methylcellulose, hydroxypropyl cellulose, methylcellulose, carboxymethylcellulose, hydroxymethylcellulose, and hydroxyethylcellulose; waxes; polyvinylacetate, polymethacrylates such as ammonio methacrylate copolymer, hydrogenated castor oil, and the like.

The rate-controlling polymer used in the delayed release layer may be selected from suitable pH-dependent polymer selected from cellulose acetate phthalate, cellulose acetate succinate, methylcellulose phthalate, hydroxypropyl methylcellulose phthalate, ethylhydroxycellulose phthalate, polyvinylacetate phthalate, polyvinyl butyrate acetate, vinyl acetate-maleic anhydride copolymer, styrene-maleic mono-ester copolymer, polymethacrylates such as methyl acrylate-methacrylic acid copolymer, methacrylate-methacrylic acid-octyl acrylate copolymer, hydrogenated castor oil, and the like. The polymer may be used either alone or in combination with other polymers for example, the enteric polymers. The enteric polymer may be selected from various pharmaceutically acceptable polymethacrylates, for example methacrylic acid co-polymers. In particular, co-polymers based on methacrylic acid and methyl methacrylate sold under the brand name EUDRAGIT® may be used. Examples include EUDRAGIT® L series (a cationic polymer synthesized from dimethylaminoethyl methacrylate) such as EUDRAGIT® L 12.5, EUDRAGIT® L 12.5P, EUDRAGIT® L 100, EUDRAGIT® L 100-55, EUDRAGIT® L-30, EUDRAGIT® L-30 D-55; the EUDRAGIT® S series such as EUDRAGIT® S 12.5, EUDRAGIT® S 12.5P, EUDRAGIT® S100; the EUDRAGIT® NE series such as EUDRAGIT® NE 30D; the EUDRAGIT® RL series such as EUDRAGIT® RL 12.5, EUDRAGIT® RL 100, EUDRAGIT® RL PO, EUDRAGIT® RL 30D; and the EUDRAGIT® RS series such as EUDRAGIT® RS 12.5, EUDRAGIT® RS 100, EUDRAGIT® RS PO, EUDRAGIT® RS 30D; and the like.

The functional layer may be applied by dispersing or suspending the rate-controlling polymer and optionally a plasticizer, anti-tacking agent and opacifying agent in a suitable medium, such as water or aqueous acidic or alkaline solutions, or in organic solvents such as methanol, ethanol, isopropanol, acetone, methyl ethyl ketone, methylene chloride, ethylene chloride, ethyl acetate, or mixtures thereof, and the resultant solution or suspension may be sprayed directly on the core or separating layer, followed by drying.

The pharmaceutical composition may be developed in the form of tablets, capsules, granules, pellets, powder, sachets, minitablets, microtablets, microspheres, microcapsules, bilayer tablets, layered tablets, tablets in a tablet, or tablets in a capsule.

The pharmaceutical compositions as described herein may comprise one or more pharmaceutically acceptable excipients selected from diluent, disintegrant, binder, lubricant, glidant, plasticizer, anti-tacking agent, opacifying agent, and the like.

Suitable diluents may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol or erythritol; and mixtures thereof. Diluent may be added to increase the bulk volume of the powder to facilitate granulation or compression.

Suitable disintegrants may include one or more of croscarmellose sodium, sodium starch glycolate, pregelatinized starch, sodium carboxymethyl cellulose, cross-linked polyvinylpyrrolidone and mixtures thereof. The disintegrant may be present in an amount ranging from 1% to 10% by weight of the composition.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like. The binder used in the separating layer is preferably water soluble binder. The binder may be present in an amount ranging from 0.1% to 10% by weight of the composition.

Suitable anti-tacking agents, lubricants or glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and mixtures thereof. It would be appreciated that a person skilled in the art is cognizant of the fact that anti-tacking agent, lubricant or glidant may be used interchangeably. The anti-tacking agent, lubricant or glidant may be present in an amount ranging from 0.1% to 20% by weight of the composition.

A suitable plasticizer may be used in a layer to increase the flexibility and strength of the layer and may include one or more of propylene glycol, polyethylene glycol, triethyl citrate, acetyl triethyl citrate, diethyl phthalate, dibutyl phthalate, dibutyl sebacate; or mixtures thereof. The plasticizer may be present in an amount ranging from 0.1% to 20% by weight of the composition.

A suitable opacifying agent may be used in a layer to prevent photo-degradation and may include one or more of titanium dioxide, iron oxides, and the like. The opacifying agent may be present in an amount ranging from 0.1% to 10% by weight of the composition.

The pharmaceutical compositions can be manufactured by various techniques or processes known to a person skilled in the art, including direct compression, dry or wet granulation, fluidized bed granulation, melts extrusion, spray drying and solution evaporation.

In one embodiment, composition of the invention may be prepared by a) preparing a core containing sugar spheres, an active substance or salts thereof and a binder; b) applying a barrier layer containing at least one sugar; and c) applying a functional layer.

The composition may contain sugar to a binder ratio in a barrier layer from 1:0.5 to 1:2, for example 1:1. The composition may contain a ratio of sugar to a binder to anti-tacking agent in a barrier layer from 80:10:10 to 20:70:10.

In another embodiment, the composition releases from about 0% to about 40% drug in vitro after 1 hour in the presence of 40% alcohol at pH 1.2. In particular, the composition releases from about 0% to about 10% drug in vitro after 1 hour in the presence of 40% alcohol at pH 1.2.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| Sr. | | Qty/cap |
|---|---|---|
| No | Ingredients | (mg) |
| | DRUG LAYER | |
| 1 | Sugar spheres | 64.00 |
| 2 | Duloxetine hydrochloride | 67.30 |
| 3 | Hypromellose | 12.70 |
| 4 | Purified water | q.s. |
| | BARRIER LAYER | |
| 5 | Sucrose | 44.80 |
| 6 | Hypromellose | 44.80 |
| 7 | Talc | 22.40 |
| 8 | Purified water | q.s. |
| | ENTERIC LAYER | |
| 9 | Hypromellose phthalate | 31.66 |
| 10 | Triethyl citrate | 3.17 |
| 11 | Talc | 3.17 |
| 12 | Isopropyl alcohol | q.s. |
| 13 | Methylene chloride | q.s. |
| | BLENDING | |
| 14 | Talc | q.s |
| | Total | 294.00 |

Procedure: Duloxetine hydrochloride was dispersed in aqueous solution of hypromellose to make drug dispersion. The dispersion was sprayed onto sugar spheres to obtain drug loaded pellets. Sucrose and hypromellose (HPMC) were dissolved in water and talc was added to obtain dispersion, this dispersion was sprayed onto drug loaded pellets to obtain barrier layered pellets. Hypromellose phthalate (HPMCP) was dissolved in a mixture of isopropyl alcohol and methylene chloride. Triethyl citrate and talc were dispersed in this solution. The dispersion was sprayed onto barrier layered pellets. These enteric coated pellets were blended with talc and filled into capsules.

TABLE 1a
Comparative Dissolution profiles of Innovator's product (Cymbalta ®
DR capsules) and Example 1
[Dissolution details: USP apparatus I, 100 RPM, 1000 mL of 0.1N HCl
with 20% ethanol]

| | |
|---|---|
| | Cumulative |
| Time | drug release (%) |

| point | | Composition |
|---|---|---|
| (min) | Cymbalta ® | of invention |
| 0 | 0.0 | 0.0 |
| 15 | 0.0 | 0.1 |
| 30 | 0.0 | 0.7 |
| 45 | 13.7 | 1.6 |
| 60 | 52.7 | 5.4 |
| 75 | 74.6 | 14.1 |
| 90 | 79.6 | 25.6 |
| 105 | 76.8 | 36.5 |
| 120 | 72.4 | 43.7 |

### Example 2

TABLE 2

| Sr. | | Qty/cap |
|---|---|---|
| No. | Ingredients | (mg) |
| | DRUG LAYER | |
| 1 | Suger spheres | 20.00 |
| 2 | Duloxetine hydrochloride | 67.30 |
| 3 | Hypromellose | 12.70 |
| 4 | Purified water | Q.s |
| | BARRIER LAYER | |
| 5 | Sucrose | 32.00 |
| 6 | Hypromellose | 32.00 |
| 7 | Talc | 16.00 |
| 8 | Purified water | Q.s |
| | FUNCTIONAL LAYER | |
| 9 | Ethyl cellulose | 18.90 |
| 10 | Hypromellose | 8.10 |
| 11 | Isopropyl alcohol | Q.s. |
| 12 | Methylene chloride | Q.s. |
| 13 | Purified water | Q.s. |
| | ENTERIC LAYER | |
| 14 | Hypromellose Phthalate | 25.88 |
| 15 | Triethyl citrate | 2.59 |
| 16 | Talc | 2.59 |
| 17 | Isopropyl alcohol | Q.s |
| 18 | Methylene chloride | Q.s |
| | BLENDING | |
| 19 | Talc | Q.s |
| | Total | 238.06 |

Procedure: Duloxetine hydrochloride was dispersed in aqueous solution of HPMC to make drug dispersion. The dispersion was sprayed onto sugar spheres to obtain drug loaded pellets. Sucrose and hypromellose (HPMC) were dissolved in water and talc was added to obtain dispersion, this dispersion was sprayed onto drug loaded pellets to obtain barrier layered pellets. Ethylcellulose and hypromellose (HPMC) were dissolved in a mixture of isopropyl alcohol and methylene chloride. This solution was coated on barrier layered pellets. Hypromellose phthalate (HPMCP) was dissolved in a mixture of isopropyl alcohol and methylene chloride. Triethyl citrate and talc were dispersed in the solution. This dispersion was sprayed onto polymer layered pellets. These enteric layered pellets were blended with talc and filled into capsules.

TABLE 2a
Comparative Dissolution profiles of Innovator's product (Cymbalta ®
DR capsules) and Example 2
[Dissolution details: USP apparatus I, 100 RPM, 1000 mL of 0.1N HCl
with 40% ethanol]

| | |
|---|---|
| | Cumulative |
| Time | drug release (%) |

| point | | Composition |
|---|---|---|
| (min) | Cymbalta ® | of invention |
| 0 | 0.0 | 0.0 |
| 15 | 13.6 | 0.5 |
| 30 | 47.6 | 0.5 |
| 45 | 69.9 | 0.5 |
| 60 | 82.8 | 3.9 |
| 75 | 88.9 | 21.8 |
| 90 | 92.8 | 41.4 |
| 105 | 93.1 | 53.8 |
| 120 | 93.3 | 61.8 |

### Example 3

TABLE 3

| Sr. | | Qty/Tab |
|---|---|---|
| No. | Ingredients | (mg) |
| | CORE TABLET | |
| 1 | Venlafaxine hydrochloride | 42.43 |
| 2 | Lactose monohydrate | 69.33 |
| 3 | Polyvinylpyrrolidone | 3.75 |
| 4 | Colloidal silicon dioxide | 0.50 |
| 5 | Magnesium stearate | 1.75 |
| | BARRIER LAYER | |
| 6 | Sucrose | 11.80 |
| 7 | Hypromellose | 11.80 |
| 8 | Talc | 5.90 |
| | FUNCTIONAL LAYER | |
| 9 | Ethyl cellulose | 8.14 |
| 10 | Hypromellose | 2.90 |
| 11 | Isopropyl alcohol | q.s. |
| 12 | Water | q.s. |
| | Total | 158.30 |

Procedure: Venlafaxine hydrochloride and lactose were sifted and mixed together. The mixture was granulated with the help of a solution of polyvinylpyrrolidone in water. The granules were dried and lubricated with colloidal silicon dioxide and magnesium stearate. The lubricated mass was compressed into tablet by using proper tooling. Sucrose and hypromellose (HPMC) were dissolved in water and talc was added to obtain dispersion, this dispersion was sprayed onto tablets. Ethylcellulose and hypromellose (HPMC) were dissolved in a mixture of isopropyl alcohol and water. This solution was coated on the barrier layered tablets.

TABLE 3a
Comparative Dissolution profiles of Innovator's product (Effexor ®
XR ER capsule) and Example 3
[Dissolution details: USP apparatus I, 100 RPM, 900 mL of 0.1N HCl
with 40% ethanol]

| | |
|---|---|
| | Cumulative |
| Time | drug release (%) |

| point | | Composition |
|---|---|---|
| (min) | Effexor ® XR | of invention |
| 0 | 0 | 0 |
| 15 | 2.2 | 0 |
| 30 | 10.5 | 0 |
| 60 | 44.3 | 0.9 |
| 90 | 65.2 | 4.1 |
| 120 | 76.2 | 10.3 |

### Example 4

TABLE 4

| | | QTY/TAB (MG) | | |
|---|---|---|---|---|
| SR. | | Example | Example | Example |
| NO. | INGREDIENTS | 4A | 4B | 4C |
| | DRUG LAYER | | | |
| 1 | Sugar Spheres | 64.00 | 64.00 | 64.00 |
| 2 | Duloxetine hydrochloride | 67.30 | 67.30 | 67.30 |
| 3 | Hypromellose 6 cPs | 12.70 | 12.70 | 12.70 |
| 4 | Purified water | QS | QS | QS |
| | BARRIER LAYER | | | |
| 5 | Sucrose | 64.80 | 86.40 | 172.80 |
| 6 | Hypromellose 6 cPs | 21.60 | 28.80 | 57.60 |
| 7 | Talc | 21.60 | 28.80 | 57.60 |
| 8 | Purified water | QS | QS | QS |
| | ENTERIC LAYER | | | |
| 9 | Hypromellose Phthalate | 31.50 | 36.0 | 54.0 |
| 10 | Triethyl citrate | 3.15 | 3.60 | 5.40 |
| 11 | Talc | 3.15 | 3.60 | 5.40 |
| 12 | Isopropyl alcohol | QS | QS | QS |
| 13 | Methylene chloride | QS | QS | QS |
| | TOTAL | 289.80 | 331.20 | 496.80 |

Procedure: Duloxetine hydrochloride was dispersed in aqueous solution of HPMC to make drug dispersion. The dispersion was sprayed onto sugar spheres to obtain drug loaded pellets. Sucrose and hypromellose (HPMC) were dissolved in water and talc was added to obtain dispersion, this dispersion was sprayed onto drug loaded pellets to obtain barrier layered pellets. Hypromellose phthalate (HPMCP) was dissolved in a mixture of isopropyl alcohol and methylene chloride. Triethyl citrate and talc were dispersed in the solution. This dispersion was sprayed onto barrier layered pellets. These enteric layered pellets were filled into capsules.
Examples 4A, 4B and 4C contain different percentage weight gain (75%, 100% and 200% respectively) of barrier coating over the drug loaded pellets. Ratio of sucrose:HPMC:talc in the barrier layer in these examples is 60:20:20.

TABLE 4a
Dissolution profiles of Examples 4A, 4B and 4C
[Dissolution details: USP apparatus I, 100 RPM, 900 mL of 0.1N HCl
with 40% ethanol]

| Time | | | |
|---|---|---|---|
| point | Cumulative drug release (%) | | |
| (min) | Example 4A | Example 4B | Example 4C |
| 15 | 7.1 | 0.5 | 0.1 |
| 30 | 30.7 | 9.2 | 0.2 |
| 45 | 43.3 | 28.6 | 7.0 |
| 60 | 47.3 | 38.8 | 23.3 |
| 90 | 49.4 | 43.4 | 37.7 |
| 120 | 49.4 | 43.0 | 42.5 |

### Example 5

TABLE 5

| SR. | | QTY/TAB |
|---|---|---|
| NO. | INGREDIENTS | (MG) |
| | DRUG LAYER | |
| 1 | Sugar Spheres | 64.00 |
| 2 | Duloxetine hydrochloride | 67.30 |
| 3 | Hypromellose 6 cPs | 12.70 |
| 4 | Purified water | QS |
| | BARRIER LAYER | |
| 5 | Sucrose | 86.40 |
| 6 | Hypromellose 6 cPs | 10.80 |
| 7 | Talc | 10.80 |
| 8 | Purified water | QS |
| | ENTERIC LAYER | |
| 9 | Hypromellose Phthalate | 31.50 |
| 10 | Triethyl citrate | 3.15 |
| 11 | Talc | 3.15 |
| 12 | Isopropyl alcohol | QS |
| 13 | Methylene chloride | QS |
| | TOTAL | 289.80 |

Procedure: Duloxetine hydrochloride was dispersed in aqueous solution of HPMC to make drug dispersion. The dispersion was sprayed onto sugar spheres to obtain drug loaded pellets. Sucrose and hypromellose (HPMC) were dissolved in water and talc was added to obtain dispersion, this dispersion was sprayed onto drug loaded pellets to obtain barrier layered pellets. Hypromellose phthalate (HPMCP) was dissolved in a mixture of isopropyl alcohol and methylene chloride. Triethyl citrate and talc were dispersed in the solution. This dispersion was sprayed onto barrier layered pellets. These enteric layered pellets were filled into capsules.
Ratio of sucrose:HPMC:talc in the barrier layer in this example is 80:10:10.

TABLE 5a
Dissolution profiles of Example 5
[Dissolution details: USP apparatus I, 100 RPM, 900 mL of 0.1N HCl
with 40% ethanol]

| Time | Cumulative drug release |
|---|---|
| point | (%) |
| (min) | Example 5 |
| 15 | 0.6 |
| 30 | 9.1 |
| 45 | 29.0 |
| 60 | 39.0 |
| 90 | 42.5 |
| 120 | 41.6 |

### Example 6

TABLE 6

| | | QTY/TAB (MG) | | |
|---|---|---|---|---|
| SR. | | Example | Example | Example |
| NO. | INGREDIENTS | 6A | 6B | 6C |
| | DRUG LAYER | | | |
| 1 | Sugar Spheres | 64.00 | 64.00 | 64.00 |
| 2 | Duloxetine hydrochloride | 67.30 | 67.30 | 67.30 |
| 3 | Hypromellose 6 cPs | 12.70 | 12.70 | 12.70 |
| 4 | Purified water | QS | QS | QS |
| | BARRIER LAYER | | | |
| 5 | Lactose | 64.80 | 86.40 | 172.80 |
| 6 | Hypromellose 6 cPs | 21.60 | 28.80 | 57.60 |
| 7 | Talc | 21.60 | 28.80 | 57.60 |
| 8 | Purified water | QS | QS | QS |
| | ENTERIC LAYER | | | |
| 9 | Hypromellose Phthalate | 31.50 | 36.0 | 54.0 |
| 10 | Triethyl citrate | 3.15 | 3.60 | 5.40 |
| 11 | Talc | 3.15 | 3.60 | 5.40 |
| 12 | Isopropyl alcohol | QS | QS | QS |
| 13 | Methylene chloride | QS | QS | QS |
| | TOTAL | 289.80 | 331.20 | 496.80 |

Procedure: Duloxetine hydrochloride was dispersed in aqueous solution of HPMC to make drug dispersion. The dispersion was sprayed onto sugar spheres to obtain drug loaded pellets. Lactose and hypromellose (HPMC) were dissolved in water and talc was added to obtain dispersion, this dispersion was sprayed onto drug loaded pellets to obtain barrier layered pellets. Hypromellose phthalate (HPMCP) was dissolved in a mixture of isopropyl alcohol and methylene chloride. Triethyl citrate and talc were dispersed in the solution. This dispersion was sprayed onto barrier layered pellets. These enteric layered pellets were filled into capsules.

Three examples 6A, 6B and 6C contain different percentage weight gain (75%, 100% and 200% respectively) of barrier coating over the drug loaded pellets. Ratio of lactose:HPMC:talc in these examples is 60:20:20 in the barrier layer.

TABLE 6a
Dissolution profiles of Examples 6A and 6C
[Dissolution details: USP apparatus I, 100 RPM, 900 mL of 0.1N HCl
with 40% ethanol]

| | |
|---|---|
| Time | Cumulative |
| point | drug release (%) |

| (min) | Example 6A | Example 6C |
|---|---|---|
| 15 | 0.1 | 0.0 |
| 30 | 2.6 | 0.0 |
| 45 | 13.7 | 1.4 |
| 60 | 25.7 | 7.3 |
| 90 | 35.1 | 22.1 |
| 120 | 37.5 | 30.6 |

### COMPARATIVE EXAMPLE

### Without Barrier Layer

TABLE 7

| SR. | | QTY/TAB |
|---|---|---|
| NO. | INGREDIENTS | (MG) |
| | DRUG LAYER | |
| 1 | Sugar Spheres | 64.00 |
| 2 | Duloxetine hydrochloride | 67.30 |
| 3 | Hypromellose 6 cPs | 12.70 |
| 4 | Purified water | QS |
| | ENTERIC LAYER | |
| 5 | Hypromellose Phthalate | 18.00 |
| 6 | Triethyl citrate | 1.80 |
| 7 | Talc | 1.80 |
| 8 | Isopropyl alcohol | QS |
| 9 | Methylene chloride | QS |
| | TOTAL | 165.60 |

Procedure: Duloxetine hydrochloride was dispersed in aqueous solution of HPMC to make drug dispersion. The dispersion was sprayed onto sugar spheres to obtain drug loaded pellets. Hypromellose phthalate (HPMCP) was dissolved in a mixture of isopropyl alcohol and methylene chloride. Triethyl citrate and talc were dispersed in the solution. This dispersion was sprayed onto drug loaded pellets. These enteric layered pellets were filled into capsules.

TABLE 7a
Dissolution profiles of Comparative Example
[Dissolution details: USP apparatus I, 100 RPM, 900 mL of 0.1N HCl
with 40% ethanol]

| | Cumulative drug release |
|---|---|
| Time point | (%) |
| (min) | Comparative Example |
| 15 | 25.7 |
| 30 | 39.0 |
| 45 | 42.3 |
| 60 | 41.6 |
| 90 | 42.2 |
| 120 | 41.0 |

Examples 4, 5 & 6 and Comparative Example-1, enteric layer is 15% by weight of the barrier layered pellets or drug loaded pellets in absence of barrier layer.

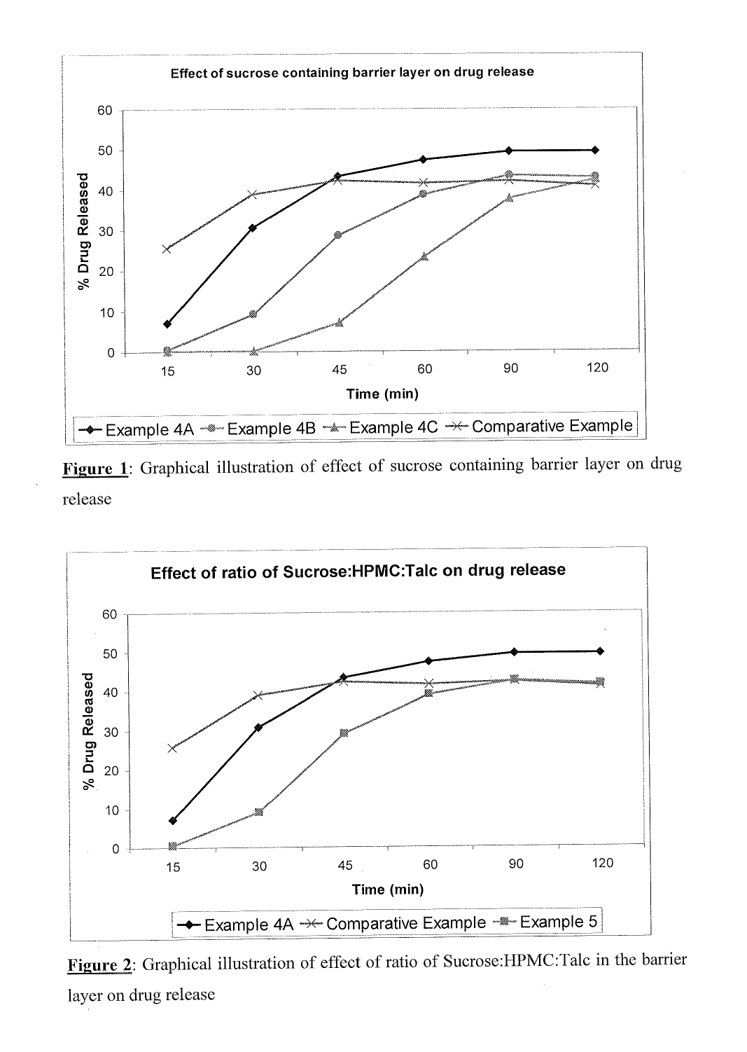

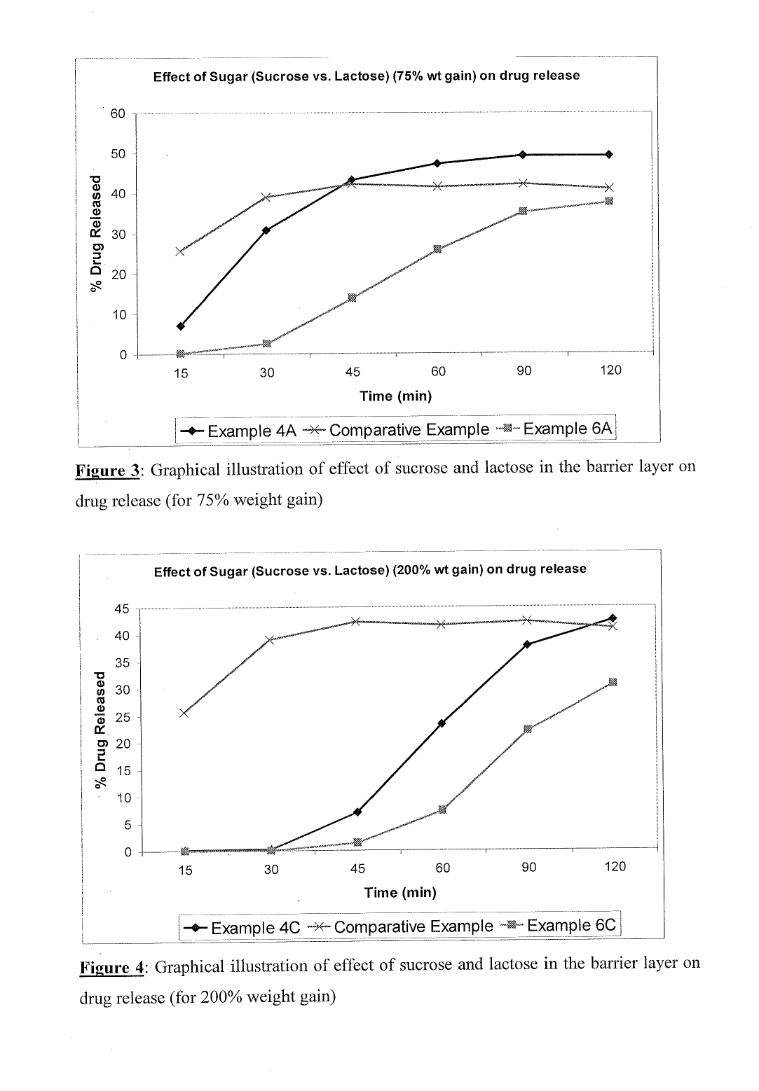

The effect of sucrose containing barrier layer on drug release illustrated in FIG. 1. The effect of different ratios of sucrose:HPMC:talc on drug release is illustrated in FIG. 2. Comparative effects of barrier layer containing sucrose and lactose in different drug loads is illustrated in FIGS. 3 & 4.

### Example 7

TABLE 8

| SR. | | QTY/TAB |
|---|---|---|
| NO. | INGREDIENTS | (MG) |
| | DRUG LAYER | |
| 1 | Acetaminophen | 600.00 |
| 2 | Hydrocodone | 10.00 |
| 3 | Microcrystalline cellulose | 108.00 |
| 4 | Povidone K-30 | 24.00 |
| 5 | Magnesium stearate | 8.00 |
| 6 | Purified water | QS |
| | BARRIER LAYER | |
| 7 | Lactose | 60.00 |
| 8 | Hypromellose 6 cPs | 7.50 |
| 9 | Talc | 7.50 |
| 10 | Purified water | QS |
| | ENTERIC LAYER | |
| 11 | Ethylcellulose | 70.00 |
| 12 | Polyethylene glycol | 12.00 |
| 13 | Isopropyl alcohol | QS |
| 14 | Dichloromethane | QS |
| | TOTAL | 907.00 |

Procedure: Acetaminophen, hydrocodone and microcrystalline cellulose were mixed together and blend was granulated with aqueous solution of povidone. The granules were dried, lubricated with magnesium stearate and compressed into tablets. Lactose and hypromellose (HPMC) were dissolved in water and talc was added to obtain dispersion, this dispersion was sprayed onto core tablets. Ethylcellulose and polyethylene glycol were dissolved in a mixture of isopropyl alcohol and dichloromethane & this solution was sprayed onto barrier layered tablets to obtain final enteric coated tablets.

From all the above examples, it can be seen that the compositions according to the invention show less drug release in the dissolution medium containing alcohol as compared to drug release from the Innovators' product or the compositions without barrier layer. Thus, the composition according to the invention are resistant to alcohol induced dose dumping.

While the invention has been described in terms of its specific embodiments, certain modifications and equivalents will be apparent to those skilled in the art and are intended to be included within the scope of the invention.

1. A pharmaceutical composition comprising a core comprising an active substance or a salt thereof; a separating layer comprising at least one sugar; and a functional layer comprising at least one pharmaceutically acceptable polymer, wherein the composition is resistant to dose dumping in presence of alcohol.

2. The composition as claimed in claim 1, wherein the separating layer further comprises a binder and an anti-tacking agent.

3. The composition as claimed in claim 1, wherein the sugar comprises one or more of sucrose, lactose, dextrin, dextrose, fructose, glucose, mannitol, sorbitol, trehalose, xylitol, isomalt, maltitol, inositol, and lactitol.

4. The composition as claimed in claim 1, wherein the sugar is sucrose.

5. The composition as claimed in claim 1, wherein the sugar is lactose.

6. The composition as claimed in claim 2, wherein the binder comprises one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, and synthetic resins.

7. The composition as claimed in claim 2, wherein the binder is hydroxypropyl methylcellulose.

8. The composition as claimed in claim 2, wherein the anti-tacking agent comprises one or more of talc, magnesium stearate, calcium stearate, zinc stearate, colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, and kaolin.

9. The composition as claimed in claim 2, wherein the anti-tacking agent is talc.

10. The composition as claimed in claim 1, wherein the separating layer comprises a sugar and a binder in a ratio of from 1:0.5 to 1:2.

11. The composition as claimed in claim 1, wherein the separating layer comprises a sugar, a binder and anti-tacking agent in a ratio of from 80:10:10 to 20:70:10.

12. The composition as claimed in claim 1, wherein the polymer is a rate controlling polymer.

13. The composition as claimed in claim 12, wherein the rate controlling polymer provides sustained release, controlled release or extended release of the active substance from the composition.

14. The composition as claimed in claim 12, wherein the rate controlling polymer provides delayed release of the active substance from the composition.

15. The composition as claimed in claim 1, wherein the polymer comprises one or more of hydrophilic polymers and hydrophobic polymers.

16. The composition as claimed in claim 1, wherein the polymer comprises one or more of ethylcellulose, hydroxypropyl methylcellulose, hydroxypropyl cellulose, methylcellulose, carboxymethylcellulose, hydroxymethylcellulose, hydroxyethylcellulose, waxes, polyvinylacetate, polymethacrylates, and hydrogenated castor oil.

17. The composition as claimed in claim 1, wherein the polymer comprises one or more of cellulose acetate phthalate, cellulose acetate succinate, methylcellulose phthalate, hydroxy propyl methylcellulose phthalate, ethylhydroxycellulose phthalate, polyvinylacetate phthalate, polyvinyl butyrate acetate, vinyl acetate-maleic anhydride copolymer, styrene-maleic mono-ester copolymer, polymethacrylates such as methyl acrylate-methacrylic acid copolymer, methacrylate-methacrylic acid-octyl acrylate copolymer, and hydrogenated castor oil.

18. The composition of claim 1, wherein the active substance is selected from the therapeutic category of drugs like anti-inflammatory agents, sedatives, hypnotics, antibiotics, antidiabetics, antihypertensives, anti-osteoporosis agents, antithrombotic agents, antivirals, antifungals, anticholinergic agents, anxiolytic agents, adrenergics, antipsychotics, anti-parkinsonism agents, anti convulsants, antiepileptics, CNS stimulants, antianginal agents, antiarrhythmics, anti-hyperlipidemic drugs, diuretics, antiasthmatics, anticoagulants, antianemia agents, vitamins, hormones, antihistaminics, anticancer agents, antiallergics, antiarthritis agents, antialzheimers' agents, vasopressin antagonists, anticonvulsants, steroids, an esthetics, thrombolytics, antacids, proton pump inhibitors, protease inhibitors, platelet aggregation inhibitors, mucolytics, antimalarials, antiemetics, laxatives, expectorants, enzymes, contraceptives, bronchodilators, antitussives, antimigraine agents, anthelmintics, and anorexiants.

19. The composition of claim 1, wherein the active substance comprises one or more of venlafaxine, duloxetine, cyclobenzaprine, quetiapine, trospium, propranolol, morphine, oxycodone, oxymorphone, amlodipine, hydrocodone, diazepam, paracetamol (acetaminophen), aspirin, ciprofloxacin, dicyclomine, celecoxib, alendronate, diacerein, acyclovir, fluconazole, epinephrine, divalproex, methylphenidate, metoprolol, fenofibrate, hydrochlorothiazide, montelukast, heparin, warfarin, hemoglobin, iron, ascorbic acid, leutinizing hormone, bicalutamide, donepezil, tolvaptan, cortisones, lidocaine, calcium carbonate, saquinavir, bromhexine, promethazine, bisacodyl, pancreatin, ethinyl estradiol, salbutamol, diphenhydramine, sumatriptan, diclofenac, metronidazole, orlistat, ibuprofen, indomethacin, ketorolac, tramadolol, oxcarbazepine, pioglitazone, rosiglitazone, miglitol, vildagliptin, sitagliptin, repaglinide, voglibose, alprazolam, chlorpromazine, cimetidine, pseudoephedrine, naproxen, piroxicam, atenolol, benazepril, captopril, lisinopril, fosinopril, enalapril, furosemide, indapamide, atenolol, felodipine, verapamil, cartenolol, carvedilol, cerivastatin, diltiazem, fluvastatin, irbesartan, candesartan, methyldopa, reserpine, bupropion, fluoxetine, paroxetine, escitalopram, sertraline, amitryptiline, imipramine, fexofenadine, clopidogrel, entacapone, levodopa, carbidopa, levetiracetam, lisinopril, losartan, lovastatin, niacin, pravastatin, ramipril, simvastatin, atorvastatin, valsartan, telmisartan, sildenafil, tadalafil, vardenafil, esomeprazole, famotidine, omeprazole, pantoprazole, rabeprazole, ranitidine, simethicone, artesunate, amodiaquine, benazepril, misoprostol, metformin, glipizide, or their pharmaceutically acceptable salts.

20. The composition of claim 1, wherein the composition further comprises one or more pharmaceutically acceptable excipients comprising diluents, disintegrants, binders, lubricants, glidants, plasticizers, anti-tacking agents, and opacifying agents.

21. The composition of claim 1, wherein the composition is in the form of a tablet, capsule, granules, pellets, powder, sachet, minitablets, microtablets, microspheres, microcapsules, bilayer tablet, layered tablet, tablet in a tablet, or tablets in a capsule.

22. The composition of claim 1, wherein the composition is a capsule.

23. A pharmaceutical composition comprising a core comprising an active substance or a salt thereof; a separating layer comprising at least one sugar; and a functional layer comprising at least one pharmaceutically acceptable polymer, wherein the composition is resistant to dose dumping in presence of alcohol and wherein less than 40% of the active ingredient is released from the composition after 60 minutes in the presence of 40% alcohol at pH 1.2.

24. A process for preparing a pharmaceutical composition, the process comprising:
(i) preparing a core comprising an active substance or a salt thereof;
(ii) applying a separating layer comprising at least one sugar on the core;
(iii) applying a functional layer comprising at least one pharmaceutically acceptable polymer on the coated core of step (ii); and
(iv) converting into a suitable finished dosage form.

25. The process as claimed in claim 24, wherein the composition further comprises one or more pharmaceutically acceptable excipients comprising diluents, disintegrants, binders, lubricants, glidants, plasticizers, anti-tacking agents, and opacifying agents.
